(19) United States
(12) Patent Application Publication
(10) Pub. No.: US 2015/0004605 A1
(43) Pub. Date: Jan. 1, 2015

(54) Biomarker and Uses Thereof

(71) Applicant: ISIS INNOVATION LIMITED, Oxford (GB)

(72) Inventor: Anthony Kong, Oxfordshire (GB)

(21) Appl. No.: 14/372,195

(22) PCT Filed: Jan. 11, 2013
(86) PCT No.: PCT/GB2013/050057

§ 371 Date: Jul. 14, 2014

(30) Foreign Application Priority Data
Jan. 13, 2012 (GB) 1200507.0

(51) Int. Cl. G01N 33/574

(52) U.S. Cl. CPC G01N 33/57496; G01N 2333/916; G01N 2800/52
USPC 435/6.11; 435/7.4

(57) ABSTRACT

The present invention provides a method of determining the prognosis of a subject with cancer, or determining the progression of a cancer, or of treating a cancer, wherein the method comprises the step of determining the expression level of PTPN9 in a cancer sample from the subject. Preferably a low level of expression of PTPN9, or no expression of PTPN9, is indicative of a poor prognosis.

This invention relates to the field of medicine, in particular of oncology. In particular, it provides a novel prognostic biomarker for human cancer and a novel biomarker for stratifying cancer patients for treatment.

Cancer is one of the most common causes of disease and death in the western world. In general, incidence rates increase with age for most forms of cancer. As human populations continue to live longer, due to an increase of the general health status, cancer will affect an increasing number of individuals. The cause of most common cancer types is still at large unknown, although there is an increasing body of knowledge providing a link between environmental factors as well as genetic factors and the risk for development of cancer.

The therapeutic care of patients with cancer is primarily based on surgery, radiotherapy and chemotherapy. It is the decision of the medical practitioner, based on the clinical presentation of the patient, which therapeutic strategy to adopt. Currently the methods to determine prognosis and/or to select patients for a particular therapy relies mainly on pathological and clinical staging. However, it is very difficult to tell from current methods the prognosis, in particular for what appears to be an early stage cancer—predicting the risk of death, the risk of recurrence and the likelihood of metastasis can be very difficult.

There is therefore a need to identify prognostic makers that can accurately distinguish between tumours associated with poor prognosis including an increased probability of metastasis, increased recurrence and decreased patient survival, from others. Such prognostic makers will allow therapies to be targeted to those most in need and those most likely to benefit.

There is also a need to stratify patients to identify those that require more aggressive therapy, typically because they have a poor prognosis, and/or to identify those that will or will not respond to a particular course of treatment. This would allow patients to be given the most appropriate treatment quickly, and would avoid the administration of costly drugs which will not be effective.

The present invention demonstrates that a reduction or loss of PTPN9 expression in cancer cells, compared to non-cancer cells, correlates with a poor patient prognosis. Wherein poor prognosis is evidenced by increased metastasis, increase recurrence and/or decreased patient survival.

The present invention also demonstrates the PTPN9 levels in cancer cells can be used to stratify cancer patients, and to allow treatment to be tailored or personalised.

As used herein the term PTPN9 refers to the enzyme tyrosine-protein phosphatase non-receptor type 9, Genbank accession number: P43378.

According to a first aspect the invention provides a method for predicting or determining the prognosis of a subject with cancer, wherein the method comprises the step of determining the expression level of PTPN9 in a cancer sample from the subject, wherein a low level of expression of PTPN9, or no expression of PTPN9, is indicative of a poor prognosis.

A poor prognosis may include one or more of decreased patient survival, an increased disease recurrence and increased metastasis.

According to another aspect the present invention provides a method of determining the prognosis of a subject with cancer comprising the steps of:
(a) determining the expression level of PTPN9 in a sample from said subject; and
(b) comparing the expression level of PTPN9 determined in step (a) with one or more reference values.

The method of determining the prognosis of a cancer may also include a method for predicting or monitoring the clinical outcome of a subject affected with a cancer.

Preferably the subject has already been diagnosed with cancer. The diagnosis of cancer may be based on an assessment of one or more of clinical presentation, pathology and other biomarker expression levels.

The subject may be a human or a non-human animal. Preferably the subject is a human. A non-human animal may include dogs, cats, horses, cows, pigs, sheep and non-human primates.

The expression level of PTPN9 may be determined by measuring the quantity of PTPN9 protein or PTPN9 mRNA.

The quantity of PTPN9 protein may be determined using any suitable method. For example by immunohistochemistry, spectrometry, western blot, ELISA, immunoprecipitation, slot or dot blot assay, isoelectric focussing, SDS-PAGE, antibody microarray, radio immuno assay (RIA), fluoroimmunoassay and combinations thereof.

The quantity of PTPN9 mRNA may be determined using any suitable method. For example by RT-PCR.

Preferably the sample is a sample of a tumour or cancer cells from the subject. The cancer may be a solid cancer or a hematopoietic cancer. Preferably the cancer is a solid cancer.

The cancer may be selected from breast cancer, a head and neck cancers, pancreatic cancer, prostate cancer, ovarian cancer, cervical cancer, lung cancer, stomach cancer, bladder cancer, endometrial cancer, colon cancer, rectal cancer, testicular cancer, leukaemia, myeloma, melanoma, vulva cancer, vagina cancer, squamous cell carcinoma of skin. Preferably the cancer is breast cancer, lung and/or a head and neck cancer. The head neck cancer may be a head and neck squamous cell cancer. The cancer may be breast cancer. The cancer may be lung cancer. The cancer may be a head and neck cancer. Preferably the cancer is a squamous cell cancer.

The sample may be a sample of tumour tissue from a breast cancer. The sample may be any sample of breast cancer tumour tissue that comprises cancer cells. The sample may be a cell or a number of cells. In one embodiment the sample may be a core from the centre or the periphery of a breast cancer tumour.

Preferably the method does not include the step of obtaining the sample from the subject.

Preferably the method comprises the step of providing a cancer sample from a subject.

Preferably a low or reduced level of PTPN9 expression, or no expression of PTPN9, in the sample, compared to a reference value, is indicative of a poor prognosis.

The reference value may be the level of expression of PTPN9 in a normal sample. The normal sample may be a sample with contains only non cancer cells. Preferably the normal sample is from the same tissue type as the cancer sample. The normal sample may be obtained from the same subject as the cancer sample or from a different subject. Levels of PTPN9 expression in the cancer sample and the normal sample may be normalised by using expression levels of proteins that are known to have stable expression levels across the cancer and normal samples, for example, GADPH or beta-actin.

In one embodiment a reduction, or a low level, or a loss of PTPN9 expression in a tumour cell or tumour tissue sample compared to a reference value is indicative of a poor prognosis for the subject.

The expression of PTPN9 in the cancer sample is considered to be reduced if the level is at least 2 fold lower than the reference level or a normal level (that is the level in a normal tissues). Alternatively the expression of PTPN9 may be considered to be reduced or low if the level is at least about 20%, 25%, 50%, 75% or more lower than a reference level or a normal level. Preferably both the level in the cancer sample and the reference level or normal level are normalised.

In an alternative embodiment PTPN9 expression levels may be scored semiquantitatively using immunohistochemistry on tumour samples based on staining intensity and distribution using the immunoreactive score (IRS) as described [Nagata et al, Cancer Cell 2004]. The scoring criteria is a composite score based on staining intensity (SI) and percentage of positive cells (PP) using the formula IRS=SI×PP. The staining intensity (SI) is determined as 0=negative; 1=weak; 2=moderate; and 3=strong and the percentage of positive cells (PP) is defined as 0, <1%; 1, 1%-10%; 2, 11%-50%; 3, 51%-80%; and 4, >80% positive cells. For IRS evaluation, about ten visual fields from different areas of each tumour are usually used. A sample may be considered to have reduced or low expression of PTPN9 if the IRS score is less than 4. Typically this will consist of mainly tumours with either no or weak staining in the majority of the tumour cells.

With regard to the term “poor prognosis” this may refer of patients with a low or reduced PTPN9 expression level, or indeed no PTPN9 expression, having a hazard ratio of greater than 50, 60, 70, 80 or more, one example a hazard ratio of 83.5 (95% CI 8.2 to 851.5) was observed. A hazard ratio of 83.5 means patients are 83.5 times more likely to die compared to high PTPN9 patients over a 5 year period.

The method of the invention may be used in combination with the detection of other cancer or prognosis markers such as one or more of tumour grade, hormone receptor status, mitotic status, tumour size, and the expression of other markers.

Preferably if normal or near normal expression levels, or even increased expression levels, of PTPN9 are observed in the cancer sample compared to a normal sample/reference sample this value may correlate with a less aggressive cancer phenotype, with a better prognosis in terms of the likelihood and length of time the patient may survive, reduced risk of the cancer recurring and spreading or metastasising.

If a poor prognosis is indicated or predicted by the PTPN9 expression levels in a sample then a more aggressive treatment regimen may be suggested, for example, with the administration of adjuvant therapy.

In some cancers the Human Epidermal Growth Factor Receptor family (collectively also known as the HER or ErbB family) has been implicated. The HER family consists of four receptor tyrosine kinases, namely EGFR, HER2 (also known as neu), HER3 and HER4 (Y. Yarden, M. X. Sliwkowski, Nat Rev Mol Cell Biol 2, 127, February, 2001). HER family proteins interact with more than 10 polypeptide ligands and binding of ligand triggers specific homo- and/or heterodimerisations of the receptor proteins. Dimerized receptors then autophosphorylate and activate downstream pathways. Among downstream signalling pathways of HER family receptors are the MAPK (ERK) and Akt (PKB) pathways, which regulate cell growth, differentiation and survival. HER receptor levels are dysregulated in various types of cancer, including breast, lung and head and neck cancer. Therefore, the action of individual HER receptors and their combinatorial effects have been intensively investigated.

Furthermore, the HER family of receptors have been the target for a number of cancer therapies. These include, but are not limited to, trastuzumab (marketed as Herceptin™) Pertuzumab, gefitinib (marketed as Iressa™), erlotinib (marketed as Tarceva™), lapatinib, neratinib, afatinib, Canertinib, PF299804 and AZD8931, all which act to inhibit receptors of the HER family.

These drugs take many years to develop and hence are very costly, also there can be many side effects of taking the drugs, it is therefore important, where possible, to administer these drugs only to those patients who would benefit from their use.

Therefore, there is a need to identify markers that can allow patients to be stratified to allow more effective treatment to be administered, money to be saved and to improve patient outcomes. This is particularly important in early stage disease. Tumour markers can be used to assess the aggressiveness of a particular breast cancer tumour and the likelihood of a particular breast cancer tumour to recur or metastasise. Hence, biomarkers in breast cancer tumours can help to plan treatment that is individualised to the particular patient and biology of a particular tumour.

In addition to determining disease aggressiveness biomarkers may also be used to indicate whether or not a particular patient will respond to a particular therapy—this would avoid unnecessary administration of drugs which are unlikely to have any significant therapeutic effect.

It is an aim of the present invention to provide a biomarker that may be used to give an indication of the types of therapies that will or will not be effective for a particular cancer.

According to a further aspect, the present invention provides a method for determining the appropriate treatment for a subject comprising the steps of:
(a) determining the expression level of PTPN9 in a sample from said subject; and
(b) using the results in (a) to determine the most appropriate therapy.

Preferably the sample is a cancer sample.

Preferably the method further includes the step of comparing the expression level of PTPN9 determined in step (a) with one or more reference values before undertaking step (b).

The skilled man will appreciate that all the preferred features discussed with reference to previous aspects or embodiments of the invention may equally be applied to this aspect of the invention.

The method may be used to offer personalised medicine solutions. In one embodiment, reduced/low or no PTPN9 expression may indicate a cancer phenotype with a poor prognosis, that is likely to be more aggressive and/or more likely to spread or metastasise and/or more likely to result in recurrence or death. Therefore, reduced or no expression of PTPN9 in a tumour sample may indicate that it would be beneficial to use one or more adjuvant therapies to target cells that may remain after mastectomy or local excision of a tumour. The adjuvant therapy may be administered locally, systemically or both.

Adjuvant therapy is treatment that is given in addition to the primary, main or initial treatment, for example mastectomy or local excision of a breast tumour. An example of adjuvant therapy is the additional treatment usually given after surgery where all detectable disease has been removed, but where there remains a statistical risk of relapse due to occult disease.

The presence of PTPN9 at normal or increased levels in a sample may provide an indication that adjuvant therapy is not required.

Similarly, the presence of PTPN9 at normal or increased levels in a sample may provide an indication that the prognosis is good.

Patients with breast cancer may be treated by removing the breast tumour by surgery (mastectomy or wide local excision), this may be followed by radiotherapy. Adjuvant therapy may be used based on prognostic and predictive factor status, including tumour size and grade, lymph node status, HER2 status, oestrogen receptor-α (ER-α) status, and menopausal status. Adjuvant chemotherapy is usually given for patients with poor prognostic factors according to ‘adjuvant online’ or ‘predict’ software, especially when it is deemed that adjuvant chemotherapy will give a survival benefit of more than 5%. In this case, the patients may be offered FEC chemotherapy, consisting of 5-Fluorouracil, Epirubicin and Cyclophosphamide with or without taxane chemotherapy (usually reserved for lymph node positive patients). Postmenopausal patients may also be offered hormone therapy, while ER-α negative patients with poor prognostic factors that warrant adjuvant chemotherapy may receive chemotherapy without hormone treatment if the patients have. In patients with HER2 positive breast cancer, they may also be offered adjuvant Trastuzumab treatment. Patients with metastatic HER2 positive breast cancer patients will usually be given Trastuzumab containing chemotherapy regimen. Following progression with Trastuzumab based treatment, lapatinib with capecitabine may be given to these patients. In addition, several clinical trials have been or are currently being conducted to assess the efficacy of various HER inhibitors with or without chemotherapy for HER2 positive breast cancer patients at different stages of disease.

In one embodiment the finding of the level of expression of PTPN9 may be used as an additional independent factor to decide whether to give a patient adjuvant therapy.

Samples which show reduced/low or no PTPN9 expression may be indicative that the cancer will be resistant to HER inhibitors. Thus reduced or no PTPN9 expression in a tumour tissue sample may indicate that the subject should not be treated with inhibitors of the HER family. In this case a suitable therapy may be radiotherapy or a chemotherapeutic agent which does not act via the HER family.

By way of contrast, if a subject has normal or increased PTPN9 expression levels in a tumour tissue sample then it may be appropriate to treat them with an inhibitor of the HER family.

Known inhibitors of the HER family include, but are not limited to, trastuzumab (marketed as Herceptin™), Pertuzumab, gefitinib (marketed as Iressa™), erlotinib (marketed as Tarceva™), lapatinib, neratinib, afatinib, Canertinib, PF299804 and AZD8931, all which act to inhibit receptors of the HER family.

The method of the present invention is preferably carried out in vitro.

In another aspect the present invention provides a method of determining the progression of cancer in a subject or monitoring the response of a subject to a particular treatment, comprising the steps of:
(a) determining the expression level of PTPN9 in a sample from said subject; and
(b) comparing the expression level of PTPN9 determined in step (a) with one or more reference values.

In one embodiment, by monitoring PTPN9 levels during treatment with a HER inhibitor a decrease or loss of PTPN9 levels can be used to indicate that the subject is starting to show resistance to the therapy and that it may be time to change the therapeutic agent, for example to a non-HER inhibitor.

The reference value may be the value in a normal sample, or it may be the value obtained from a sample from the subject taken prior to a particular treatment or just earlier in disease progression.

If a subject is responding to a particular therapy, then an increase in PTPN9 levels may be expected to be observed, in particular in cancers where the PTPN9 started at a reduced level.

The invention further provides the use of PTPN9 as a prognostic biomarker in cancer.

The invention also provides the use of PTPN9 expression levels as a marker for monitoring the response of a subject with cancer to a particular treatment.

In another aspect the present invention provides a kit for use in determining the prognosis of a subject with cancer, for selecting the most appropriate therapy for a subject, and for monitoring cancer progression or response to a particular therapy, wherein the kit comprises at least one agent for determining the expression level of PTPN9 in a tumour tissue sample provided by the subject.

Preferably the kit may provide an indication useful in predicting the likelihood of a cancer spreading or recurring or of death. In one embodiment the kit may provide an indication useful in determining whether a tumour has a low risk or a high risk of spreading or recurring or death.

In another aspect the present invention provides a kit for use in determining the best treatment of cancer in a subject comprising at least one agent for determining the expression level of PTPN9 in a tumour tissue sample provided by the subject.

In one embodiment the kit may provide an indication useful in determining whether a patient should be treated using adjuvant therapy after removal of a tumour.

Preferably the agent is an antibody.

Preferably the kit may further comprise instructions for suitable operational parameters in the form of a label or separate insert.

Preferably the kit may further comprise one or more PTPN9 protein samples to be used as a standard(s) for calibration and comparison.

In another aspect the present invention provides the use of the level of PTPN9 expression in a tumour tissue sample as a biomarker to determine the prognosis of a subject with breast cancer.

In another aspect the present invention provides a use of the determination of the expression level of PTPN9 in a tumour tissue sample as a means of assessing the prognosis of an individual with breast cancer.

In a further aspect the present invention provides a method of treating cancer comprising:
i) obtaining the PTPN9 expression levels in a sample from a subject;
ii) administering treatment for the cancer based on the PTPN9 levels observed.

In step i) the PTPN9 expression levels may be obtained directly by the person administering the treatment, or the person administering the treatment may obtain the expression levels by instructing a third party to determine the PTPN9 levels. The PTPN9 levels may be obtained from a test laboratory.

In step ii) if the PTPN9 levels observed in step i) are reduced compared to a control the treatment administered may be more aggressive. For example, adjuvant therapy may be administered. This may be in addition to surgery and/or radiotherapy. If the cancer is breast cancer, and the PTPN9 levels are reduced, preferably a HER inhibitor is not administered.

Alternatively, in step ii) if the PTPN9 levels observed in step i) are substantially the same or increased compared to a control the treatment administered may be less aggressive. For example, adjuvant therapy may not be administered. The subject may be administered only surgery and/or radiotherapy. If the cancer is breast cancer, and the PTPN9 levels are substantially the same or increased, a HER inhibitor may be administered.

In a still further aspect the present invention provides a method of stratifying cancer patients into those requiring aggressive therapy and those not requiring aggressive therapy, the method comprising:
i) obtaining the PTPN9 expression levels in a sample from a subject;
ii) stratifying the subject into a group requiring aggressive therapy if the PTPN9 levels are reduced compared to a control, or into a group requiring less aggressive therapy if the PTPN9 levels are substantially the same or increased compared to a control.

The skilled man will appreciate that preferred features of any one embodiment and/or aspect of the invention may be applied to all other embodiments and/or aspects of the invention.

There now follows, by way of example only, a detailed description of the present invention with reference to the accompanying drawings, in which;

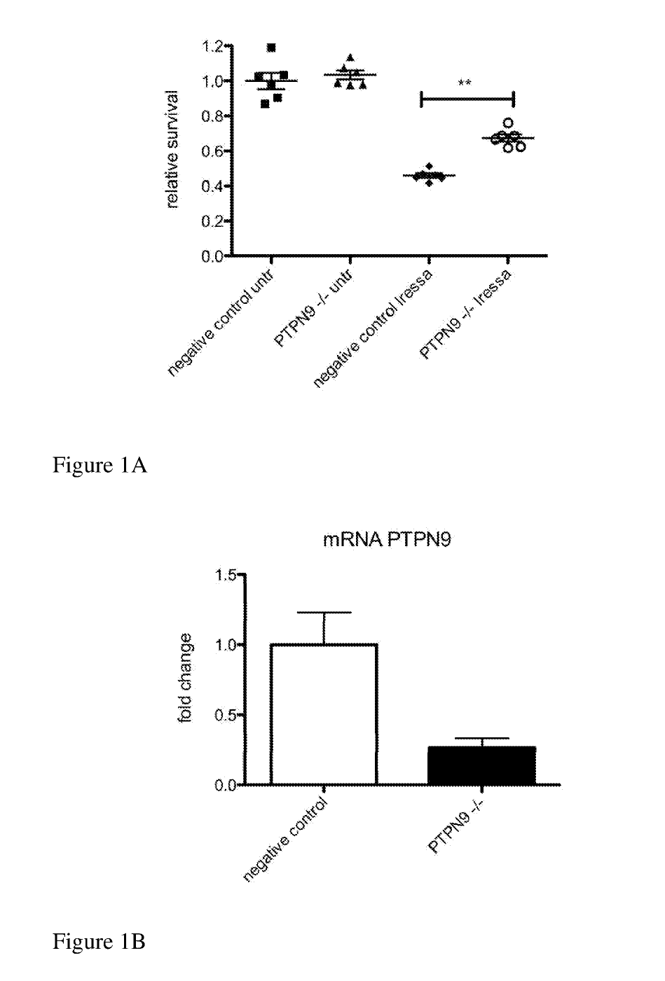

FIG. 1A to 1C—show that a loss of PTPN9 expression leads to a resistance to gefitinib (marketed as Iressa™) in HER2 positive breast cancer cells (FIGS. 1A and 1B) and head and neck squamous cell cancer cells (FIG. 1C);

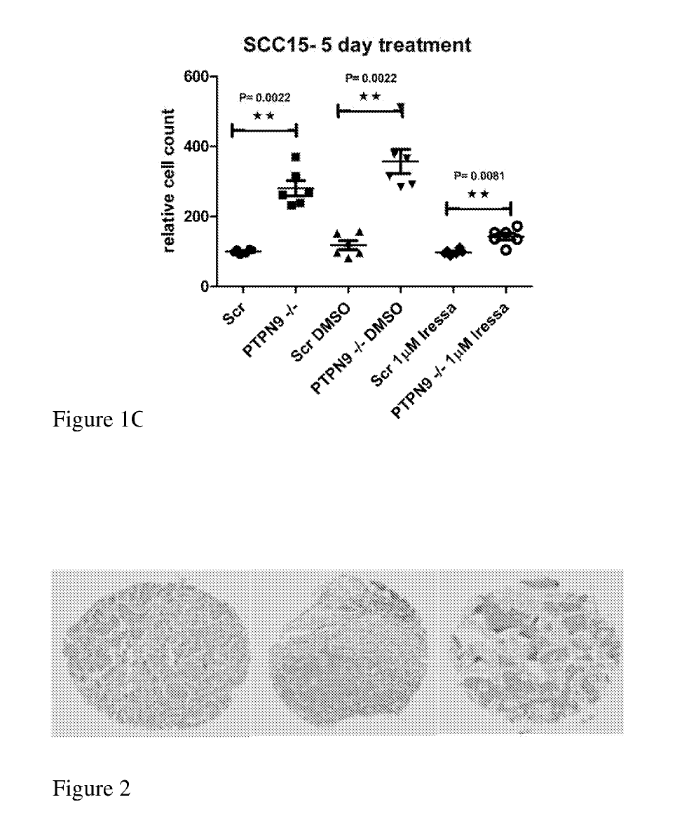

FIG. 2—shows that PTPN9 expression levels vary in different breast cancer samples from different patients;

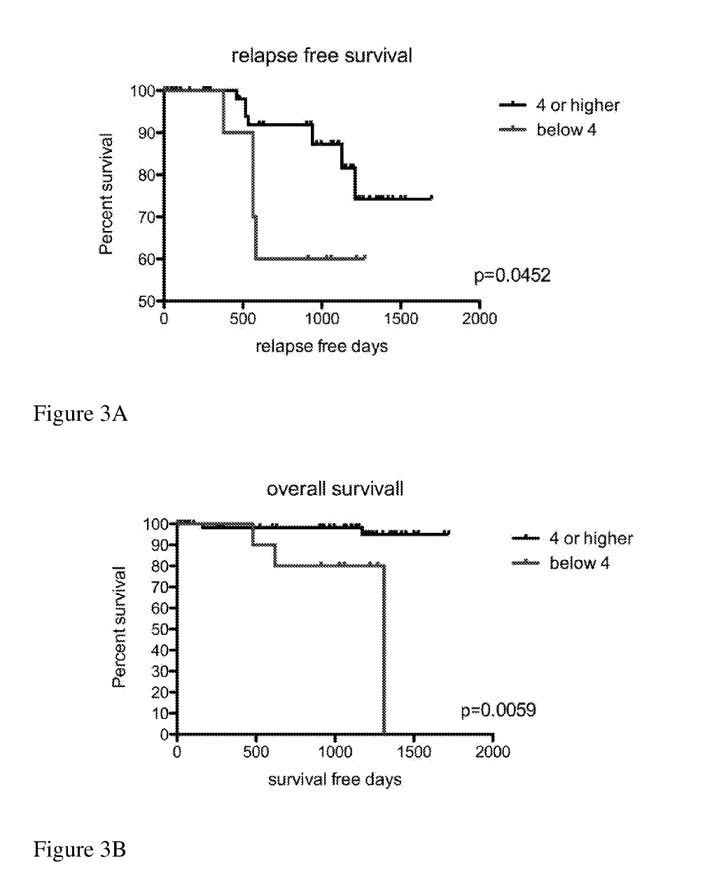

FIGS. 3A and 3B—show that PTPN9 expression levels are prognostic, more specifically that a reduction in PTPN9 levels correlates with reduced relapse free survival and reduced overall survival;

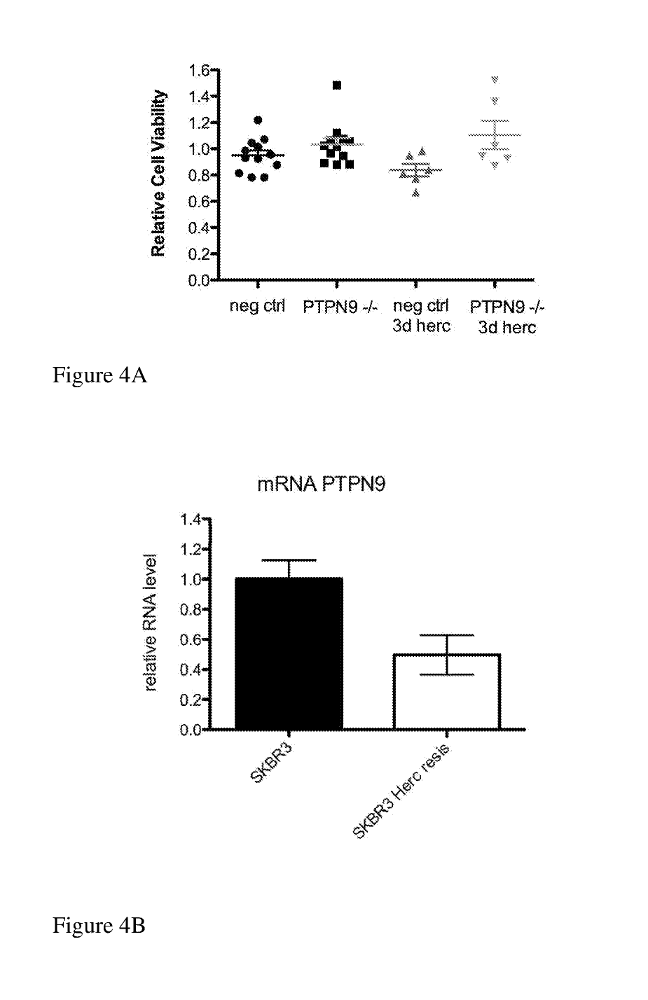

FIGS. 4A, 4B and 4C—show that a loss of PTPN9 expression leads to a slightly increased proliferation as well as resistance to trastuzumab (marketed as Herceptin™) in HER2 positive breast cancer cells (FIGS. 4A and 4B) and head and neck squamous cell cancer cells (FIG. 4C). In trastuzumab resistant cells siRNA of PTPN9 does not have much effect since there is already a loss of PTPN9 in these cells.

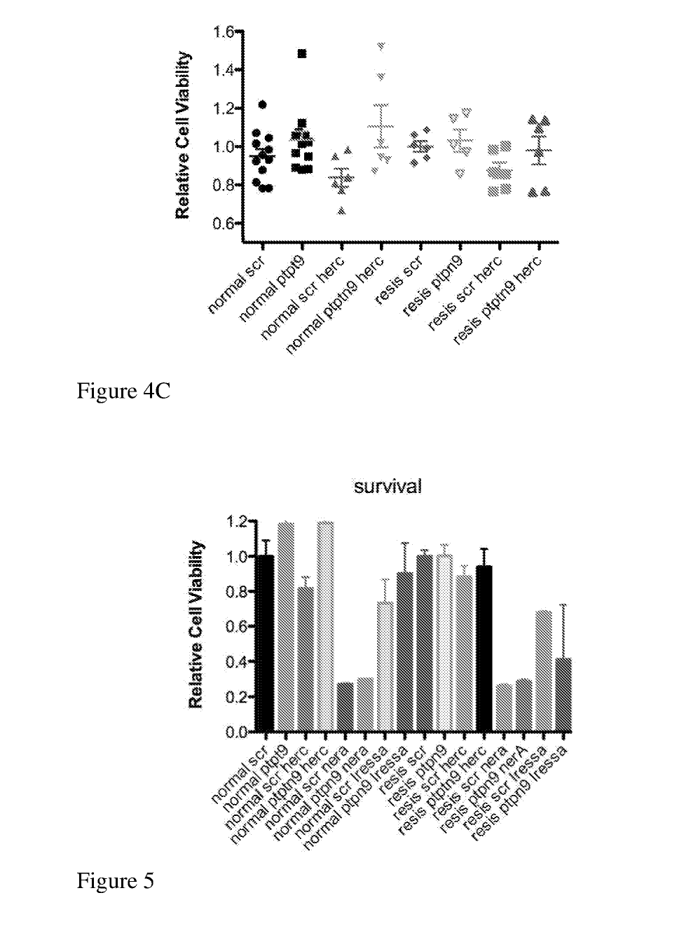

FIG. 5—shows that a loss of PTPN9 leads to decreased sensitivity to Neratinib in both sensitive and Herceptin resistant cells.

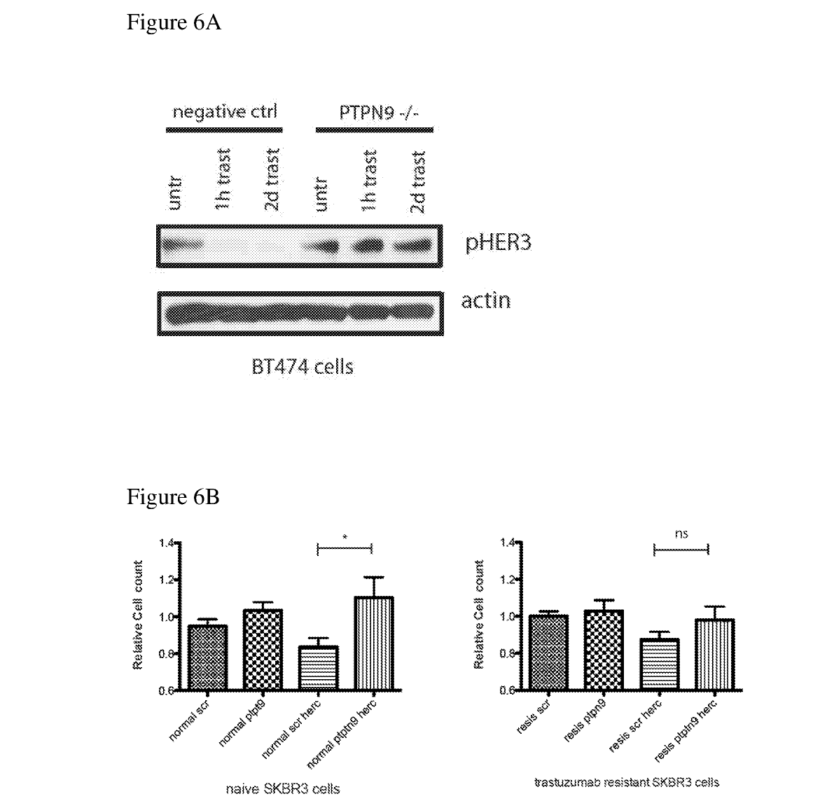

FIGS. 6A and 6B—show that cells with reduced or no PTPN9 expression are more resistant to HER inhibitors such as trastuzumab. FIG. 6A shows the results of breast cancer BT474 cells transfected with either a negative control siRNA or PTPN9 siRNA (20 nM) and then plated and treated for 1 hour or 2 days with 40 ug/ml trastuzumab. Cells were then lysed and equal amounts of protein were loaded on a NuPAGE gel. Samples were analysed by western blot for phospho-HER3 and PTPN9 levels. Actin acted as a loading control. In FIG. 6B naïve SKBR3 cells (left) or Trastuzumab resistant SKBR3 cells (right) were transfected with either a negative control siRNA or PTPN9 siRNA (20 nM). They were then plated and treated for 3 days with 40 ug/ml Trastuzumab. Remaining cells were counted using a cell counter.

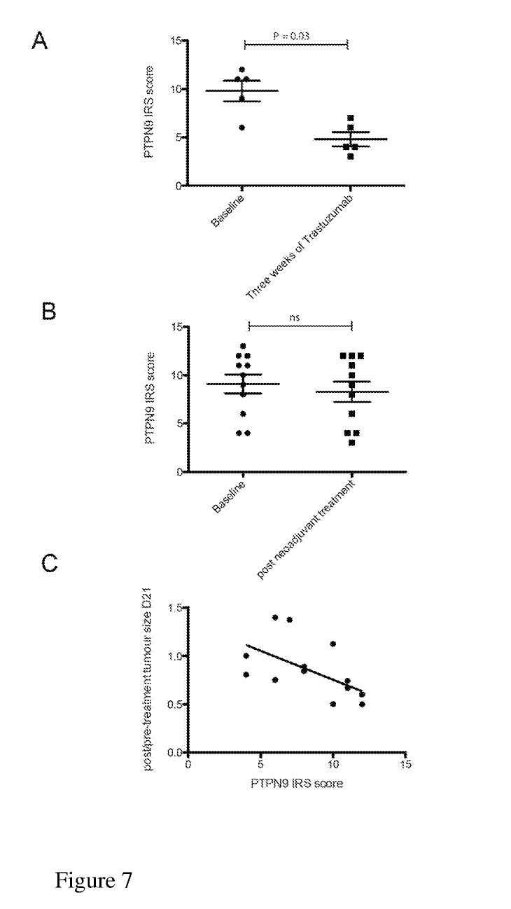

FIGS. 7A, 7B and 7C—shows that trastuzumab monotherapy decreases PTPN9 levels and that PTPN9 level is correlated with trastuzumab response in HER2 positive breast cancer patients. In particular, FIG. 7A shows the levels on PTPN9 observed in HER2 positive breast cancer patients who have been given one dose of trastuzumab (8 mg/kg) followed by 4 cycles of neoadjuvant docetaxel chemotherapy 100 mg/m² q21 with 6 mg/kg trastuzumab before surgery. Paired tissue samples (pre- and post-treatment) at day 21 after trastuzumab (8 mg/kg) monotherapy window study, were stained for PTPN9 expression, and the results of analysis of the tissue is given in FIG. 7A. FIG. 7B shows the results of the analysis of paired tissue samples (pre- and post-treatment) after further neoadjuvant chemotherapy and trastuzumab treatment. In FIG. 7C basal PTPN9 levels were correlated with clinical response (post/pre-treatment tumour size) at day 21. Scatter plots showing the relationship between the post/pre-treatment tumour size and basal PTPN9 levels and their relationships were examined using the Spearman-Rank correlation.

### LOSS OF PTPN9 INDUCES RESISTANCE TO TARGETED THERAPIES

The results presented herein demonstrate that knockdown of PTPN9 decreases sensitivity of HER2 positive breast cancer cells to Iressa™ (FIGS. 1A and 1B) and trastuzumab. In FIGS. 1A and 1B experimental breast cancer cells SKBR3 were treated with nonsense siRNA or siRNA against PTPN9 in combination with Iressa treatment for 3 d before cell viability experiments were undertaken. The results show that the SKBR3 cells were more sensitive to Iressa™ after three days than the control.

FIG. 1C shows that knockdown of PTPN9 increases proliferation as well as further decreases Iressa™ sensitivity in HER2 expressing HNSCC15 cells. HNSCC15 cells are cells derived from a head and neck squamous cell carcinoma. In this experiment HNSCC15 cells were treated with nonsense siRNA or siRNA against PTPN9 in combination with Iressa treatment for 5 d before cell viability experiments were undertaken.

### Loss of PTPN9 is Prognostic of HER2 Positive Breast Cancer

To further understand the prognostic value of PTPN9 deficiency in HER2 positive breast tumours, immunohistochemistry (IHC) was used to stain a set of HER2 positive breast tumours. PTPN9 IHC staining was optimised in BT474 cell pellets with or without siRNA against PTPN9 and showed that the antibody was specific enough to assess PTPN9 expression levels. The difference in PTPN9 expression between different tumours of the cohort of patients is shown in FIG. 2.

Using the established IRS scoring system for PTEN (Y. Nagata et al., Cancer Cell 6, 117, August, 2004), patients with PTPN9 deficiency (reduced PTPN9 expression) were shown to a have a poorer prognosis due to having poorer relapse-free survival (FIG. 3A) and poorer overall survival (FIG. 3B). The scoring criteria is composite score based on staining intensity (SI) and percentage of positive cells (PP) using the formula IRS=SI×PP (see above). An IRS score of 4 or higher was considered normal, and a score of 4 or below 4 was considered a reduced level. The overall and disease-free survival between these two groups of patients was assessed using Log-rank test and Gehan-Breslow-Wilcoxon tests.

The results show that patients with low PTPN9 had a relapse-free survival of 63% compared to high PTPN9 patients with 75% over the monitored period (p=0.045 for Gehan-Breslow-Wilcoxon test and p=0.098 for Log-rank test) (FIGS. 3A and 3B). For overall survival, the survival fraction was 75% for low PTPN9 patients compared to 93% for high PTPN9 patients (p=0.0204 for Gehan-Breslow-Wilcoxon test and p=0.0033 for Log-rank test) (FIGS. 3A and 3B). Thus, PTPN9 deficiency renders the cells more resistant to Iressa™ and was a prognostic marker for poorer relapse-free and overall survivals in these patients.

FIGS. 4A, 4B and 4C show the same results are observed when Herceptin™ (trastuzumab) is used instead of Iressa™

FIG. 5 shows that a loss of PTPN9 expression also results if resistance to neratinib.

### Loss of PTPN9 Decreases Trastuzumab Response in HER2 Positive Breast Cancer Cells

The acute and chronic effect of Trastuzumab on PTPN9 and pHER3 was assessed. The results obtained showed that acute Trastuzumab treatment increased PTPN9 level while decreasing pHER3 in SKBR3 and BT474 cells. However, with prolonged Trastuzumab treatment, PTPN9 expression decreased in both SKBR3 and BT474 cells. Comparing the naïve and resistant SKBR3 cells without Trastuzumab treatment, PTPN9 mRNA and protein levels were downregulated in resistant cells (FIGS. 4A and 4B). To assess whether pHER3 is definitely regulated by PTPN9 in HER2 positive breast cancer cells, PTPN9 knockdown was optimised and confirmed by mRNA. The results show that PTPN9 knockdown prevents Trastuzumab from decreasing HER3 phosphorylation (FIG. 6A). Furthermore, PTPN9 knockdown decreases the inhibitory effects of Trastuzumab in naïve cells and not in resistant cells (FIG. 6B, left and right panels). Collectively, the results suggested that PTPN9 regulates HER3 phosphorylation during Trastuzumab treatment and loss of PTPN9 may render the cells resistant to Trastuzumab treatment in HER2 positive breast cancer cells.

PTPN9 is Decreased after Trastuzumab and PTPN9 Level is Correlated with Trastuzumab Clinical Response

PTPN9 levels in HER2 positive breast cancer patients were assessed as the patients underwent a window study and were given one dose of Trastuzumab (8 mg/kg) followed by 4 cycles of neoadjuvant docetaxel chemotherapy 100 mg/m2 q21 with 6 mg/kg Trastuzumab before surgery. The data from paired samples of patient biopsies (5 paired of pre- and post-Trastuzumab monotherapy treatment samples are available) showed that PTPN9 levels were significantly decreased at day 21 after one dose of Trastuzumab monotherapy (FIG. 7A), similar to the cell line results. However, after neoadjuvant docetaxel chemotherapy with Trastuzumab when most of the tumours have responded, there was no difference in PTPN9 levels (FIG. 7B). The clinical significance of pre-treatment PTPN9 expression was assessed in these patients. The results showed that there was a correlation of PTPN9 with clinical response (decreased post/pre-treatment tumour size) after one dose of Trastuzumab at day 21 (R²=0.29, p=0.047) (FIG. 7C). There was a greater decrease of tumour size for patients with higher PTPN9 levels. However, there was no correlation of basal PTPN9 levels with tumour size response at definitive surgery after further 4 cycles of neodjuvant docetaxel chemotherapy with Trastuzumab (data not shown).

### CONCLUSION

The results presented herein demonstrate the prognostic value of PTPN9 and the value of PTPN9 for patient stratification to target the therapy given. In particular the data shows the role of PTPN9 as a prognostic and predictive biomarker in HER2 positive breast cancer patients.

### Materials and Methods

Detailed information on cell culture and reagents, western blot, transfection with siRNA and immunoprecipitation can be found in previous publication (K. P. Gijsen M, Perera T, Parker P, Harris A, Larijani B, Kong A, PLoS Biology 8(12), e1000563 2010), this reference is herein incorporated in its entirety. A summary of materials and methods is provided below.

### Immunofluorescence

SKBR3 cells (5×10⁴/well) grown on coverslips were treated with Akti and fixed with chilled methanol for 20 min at −20° C., and then blocked with PBS-T containing 10% FBS for 30 min at 4° C. The fixed samples were incubated overnight at 4° C. with monoclonal anti-PTPN9 antibody (D-5) (Santa Cruz, and then incubated with Alexa Fluor 488-conjugated goat anti-rabbit IgG (1:500) and Alexa Fluor 546-conjugated goat anti-mouse IgG (1:1000) (Invitrogen) for a further 2 h. Finally, the samples were washed three times with milli-Q water and mounted with Fluoromount-G (SouthernBiotech) and observed by using Axioskop2 plus fluorescence microscope (Carl ZEISS). The images of Alexa Fluor 488 and 546 were assigned as green and red respectively, and were merged together by using the ImageJ software (NIH).

### Immunohistochemistry

The xenograft sections were deparaffinised in 2 changes of citrate solution (5 minutes each) and then hydrated in 2 changes of 100% ethanol (5 minutes each). This was followed by 50% ethanol for 5 minutes and then rinsed in distilled water. Antigen retrieval was done by heating the slides in citrate buffer (10 mM citric acid, 0.05% Tween 20, pH 6.0) for 2 minutes at 125° C. and 10 min at 85° C. Following antigen retrieval, the sections were rinsed in PBS before staining them with primary antibody PTPN9 diluted in RPMI medium overnight at 4° C. After rinsing in PBS twice, the sections were incubated with peroxidase-linked anti-rabbit Ig (ImmPRESS) for 30 minutes at room temperature. Then the slides were rinsed in PBS twice and incubated in 3,3′-Diaminobenzidine solution for 5 minutes. A counterstaining was performed by incubating in hematoxylin QS solution (Vector Laboratories) for 30 seconds. Sections are mounted using an aqueous mounting solution (Aquatex).

### Cell Culture and Reagents

BT474 and SKBR3 cell lines were obtained from the London Research Institute. BT474 cells were maintained in RPMI (Gibco) supplemented with 10% FBS (PAA Laboratories), 10 μg/ml insulin (Sigma-aldrich), 100 units/ml penicillin and 100 μg/ml streptomycin (Gibco) in a humidified atmosphere containing 5% CO₂ at 37° C. SKBR3 cell were maintained in DMEM (Gibco) medium supplemented with 10% FBS, 100 units/ml penicillin and 100 μg/ml streptomycin in a humidified atmosphere containing 5% CO₂ at 37° C. Akt inhibitor VIII was purchased from Calbiochem and was dissolved in dimethyl sulfoxide (DMSO) (Sigma-aldrich) to 2.5 mM stock and stored in −20° C. Wortmannin was purchased from Sigma-aldrich and was dissolved in DMSO to 10 mM stock and stored in −20° C. PTP-N9 siRNA (sc-44670) was purchased from Thermo, and the negative universal control (45-2001) was purchased from Invitrogen.

### Western Blot

Cells were grown in 100 mm dishes and treated with either Akt inhibitor VIII, Iressa or DMSO for the indicated times and concentrations. The cells were then lysed using an ice-cold lysis buffer (20 mM Tris, pH 7.5, 10 mM EDTA, 15 mM NaCl, 10 mM Na₂H₂P₂O₇, 100 nM NaF) supplemented with complete protease inhibitor cocktail (Roche). Lysates were centrifuged at 13,200 rpm for 10 minutes at 4° C. and the supernatants were collected and assessed for protein concentration using the Bio-rad Protein Assay (Bio-rad). Standard western blot procedure was performed which can be found in previous publication (K. P. Gijsen M, Perera T, Parker P, Harris A, Larijani B, Kong A, PLoS Biology 8(12), e1000563 2010). Membranes were blotted with the following primary antibodies: rabbit monoclonal anti-phospho-HER3 (Tyr1289), rabbit polyclonal anti-phospho-Akt (Ser473), rabbit polyclonal anti-Akt, rabbit polyclonal anti-phospho-p44/42 MAPK (Thr202/Tyr204), rabbit polyclonal anti-p44/42 MAPK and rabbit monoclonal anti-β-actin (13E5) (Cell Signalling), anti-phospho-EGFR (ab40815) and anti-HER3 (ab34641) (abcam), and anti-PTP-N9 (D-5) (Santa Cruz). Antibodies were incubated in phosphate-buffered saline-Tween buffer (PBS, 0.2% Tween 20) with 3% bovine serum albumin (Sigma-Aldrich). Primary antibodies were probed with secondary horseradish-peroxidase linked anti-rabbit IgG or anti-mouse IgG (Invitrogen) in PBS, 0.2% Tween 20 with 5% non-fat dry milk. Protein-antibody complexes were detected by chemiluminescence with the Amersham ECL Detection Reagents (GE Healthcare). The experiments were repeated at least three times.

Transfection with siRNA

Cells were seeded at a density of 1×10⁵ cells/ml on the plate before the day of transfection. A given amount of each siRNA was mixed with Lipofectamine 2000 (Invitrogen) for 20 min at room temperature according to the manufacturer's instructions. The mixtures were then applied to the cells in serum-free DMEM giving a final concentration of siRNA at 100 nM. After incubation for 24 h at 37° C., DMEM supplemented with serum and antibiotics was added. The cells were then cultured for an additional 48 h at 37° C. before further analysis.

### Immunoprecipitation

Protein lysates of the treated cells (3×10⁶ per dish) or conditional medium were collected with the method as the Western blot section. The collected cell lysate (800 μg) or equal volume of collected medium was incubated with Dynabeads Protein G (Invitrogen), which was bound with appropriated antibody according to the manufacturer's instructions for 2 h at room temperature, and then eluted with 80 ml of 1× NuPAGE LDS Sample Buffer (Invitrogen) by boiling at 90° C. for 10 min. The samples were eventually analyzed by Western blot assay.

1-3. (canceled)

4. A method for determining the appropriate treatment for a subject comprising the steps of:
(c) determining an expression level of an enzyme tyrosine-protein phosphatase non-receptor type 9 (PTPN9) in a sample from said subject; and
(d) using the expression level to determine the most appropriate therapy.

5. The method of claim 4 further comprising the step of comparing the expression level of PTPN9 determined in step (c) with one or more reference values before undertaking step (d).

6. The method of claim 4 wherein a reduced level of PTPN9 expression or no PTPN9 expression is indicative that the cancer will be resistant to Human Epidermal Growth Factor Receptor (HER) inhibitors.

7. The method of claim 4 wherein a normal or increased level of PTPN9 expression is indicative that the cancer will be responsive to Human Epidermal Growth Factor Receptor (HER) inhibitors.

8. A method of determining a prognosis of a subject with cancer, determining a progression of cancer in a subject, or monitoring a response of a subject to a particular treatment for cancer, the method comprising the steps of:
(a) determining an expression level of an enzyme tyrosine-protein phosphatase non-receptor type 9 (PTPN9) in a sample from said subject; and
(b) comparing the expression level of PTPN9 determined in step (a) with a reference value.

9. The method of claim 8 wherein the reference value is a value obtained from a sample taken prior to a particular treatment or earlier in disease progression.

10. The method of claim 8 wherein the expression level of PTPN9 is determined by measuring the quantity of PTPN9 protein and/or PTPN9 mRNA.

11. The method of claim 8 wherein the sample is a cancer sample.

12. The method of claim 8 wherein the sample is a sample of a solid cancer.

13. The method of claim 8 wherein the cancer is selected from breast cancer, a head and neck cancers, pancreatic cancer, prostate cancer, ovarian cancer, cervical cancer, lung cancer, stomach cancer, bladder cancer, endometrial cancer, colon cancer, rectal cancer, testicular cancer, leukaemia, myeloma, melanoma, vulva cancer, vagina cancer and squamous cell carcinoma of skin.

14. The method of claim 13 wherein the cancer is breast cancer, lung cancer and/or a head and neck cancer.

15. The method of claim 25 wherein a low level of expression of PTPN9 in the cancer sample is a reduction in the level of at least 2 fold compared to a normal level or a reference level.

16. The method of claim 25 wherein a low level of expression of PTPN9 in the sample is a reduction in the level of at least about 20% compared to a reference value.

17. The method of claim 25 wherein a low level of expression of PTPN9 is an immunoreactive score (IRS) level of less than 4.

18. The method of claim 8 wherein the method is carried out in vitro.

19-20. (canceled)

21. A kit for use in determining the prognosis of a subject with cancer, and/or for selecting the most appropriate therapy for a subject, and/or for monitoring cancer progression or response to a particular therapy, wherein the kit comprises at least one agent for determining the expression level of an enzyme tyrosine-protein phosphatase non-receptor type 9 (PTPN9) in a tumour tissue sample provided by the subject.

22-23. (canceled)

24. A method of treating cancer comprising:
(a) determining the expression level of an enzyme tyrosine protein phosphatase non-receptor type 9 (PTPN9) in a cancer cell;
(b) determining whether the expression level is high, low or normal;
(c) administering a Human Epidermal Growth Factor Receptor (HER) inhibitor if the PTPN9 expression is normal or high, and administering a non-HER inhibitor if the PTPN9 expression is low.

25. The method according to claim 8, wherein a low level of expression of PTPN9 or no expression of PTPN9 is indicative of a poor prognosis.
